(19) United States
(12) Patent Application Publication
(10) Pub. No.: US 2012/0211385 A1
(43) Pub. Date: Aug. 23, 2012

(54) MEDICAL PRODUCT PACKAGE

(76) Inventor: Nancy Gail Limback, Valley Park, MO (US)

(21) Appl. No.: 13/029,572

(22) Filed: Feb. 17, 2011

(51) Int. Cl. A61B 19/02

(52) U.S. Cl. 206/438

(57) ABSTRACT

A package for accommodating, storing, displaying and transporting a medical product. The package has a body formed by a plurality of panels, and at least one of the panels has a first layer and a second layer. A tab is formed integrally with the first layer. The tab is pivotable via at least one hinge formed in the first layer from a first position substantially coplanar with the first layer to a second position where the tab is disposed at an angle to the first layer.

### BACKGROUND OF THE INVENTION

The present disclosure relates generally to a package for accommodating, storing, displaying and transporting medical products such as, for example, pharmaceutical products.

Packages, such as cartons, are commonly used in the pharmaceutical industry to store, display, and ship medical products as well as exhibit information related to the products. Typically, such packages can be provided with a tab depending from a side of the package, through which the package can be pegged to a board or wall for display. Meanwhile, it is desirable that the packages are designed to avoid structures like tabs permanently protruding from the package, so that the package can be packed in a compact and stacked manner without the tabs occupying space and interfering with each other. Thus, waste of limited space during the transportation, storage, and display of the packages can be avoided.

It is further desirable for the packaging, particularly in the area of medical products, to discourage and/or provide evidence of tampering. That is, a package should ensure that, absent evidence (e.g., visual, tactile, etc.) of tampering, the product within the package has not been altered since it left the manufacturer. Similarly, it is desirable that the products contained within such packages not be accessible by anyone but the end user. For example, the packages should not contain openings through which the products within the packages may be touched or handled, or through which dust, dirt or other debris may enter and potentially collect within the package.

Therefore, it is desirable to provide a novel package for medical products, which can be hung or pegged, without compromising the packability of the package during storage and transportation, and which discourages and/or provides evidence of tampering, as well as protects the contents of the package from undesirable handling or debris.

### BRIEF DESCRIPTION OF THE INVENTION

As described herein, the exemplary embodiments of the present invention overcome one or more of the above and other disadvantages known in the art.

An exemplary aspect of the present invention relates to package for a medical product. The package includes a body including a plurality of panels, wherein at least one panel includes a first layer and a second layer. The package further includes a tab formed integrally with the first layer, wherein the tab is pivotable via at least one hinge formed in the first layer from a first position substantially coplanar with the first layer to a second position where the tab is disposed at an angle to the first layer.

For example, the tab is connected to the first layer via a pair of hinges.

For example, the at least one hinge includes a score.

For example, when the tab is in the first position, the tab is connected to the first layer by a confining mechanism. For example, the confining mechanism includes at least one pre-perforated seam.

For example, the first layer is substantially planar and the tab is substantially planar.

For example, the plurality of panels are connected to provide an enclosed structure defining a space therein. For example, when the tab is in the first and second positions, the space is not accessible from an environment external to the enclosed structure.

For example, the tab includes an opening formed therein. For example, the opening includes a filler element. For example, a periphery of the opening is perforated to enable removal of the filler element.

For example, the first layer is secured to the second layer by an adhesive.

For example, the first layer includes an adhesive applied to an underside thereof for adhering to an upper surface of the second layer. For example, the adhesive covers substantially all of the underside of the first layer. For example, the adhesive covers predetermined portions of the underside of the first layer.

### BRIEF DESCRIPTION OF THE DRAWINGS

These and other aspects and advantages of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. Moreover, the drawings are not necessarily drawn to scale and, unless otherwise indicated, the drawings are merely intended to conceptually illustrate the structures and procedures described herein.

FIG. 1 is a perspective view a package for a medical product according to an exemplary embodiment of the present invention, wherein a pivotable tab of the package is shown in a first position; and

FIG. 2 is a perspective view a package for a medical product according to an exemplary embodiment of the present invention, wherein the pivotable tab is shown in a second position.

### DETAILED DESCRIPTION OF THE EXEMPLARY EMBODIMENTS OF THE INVENTION

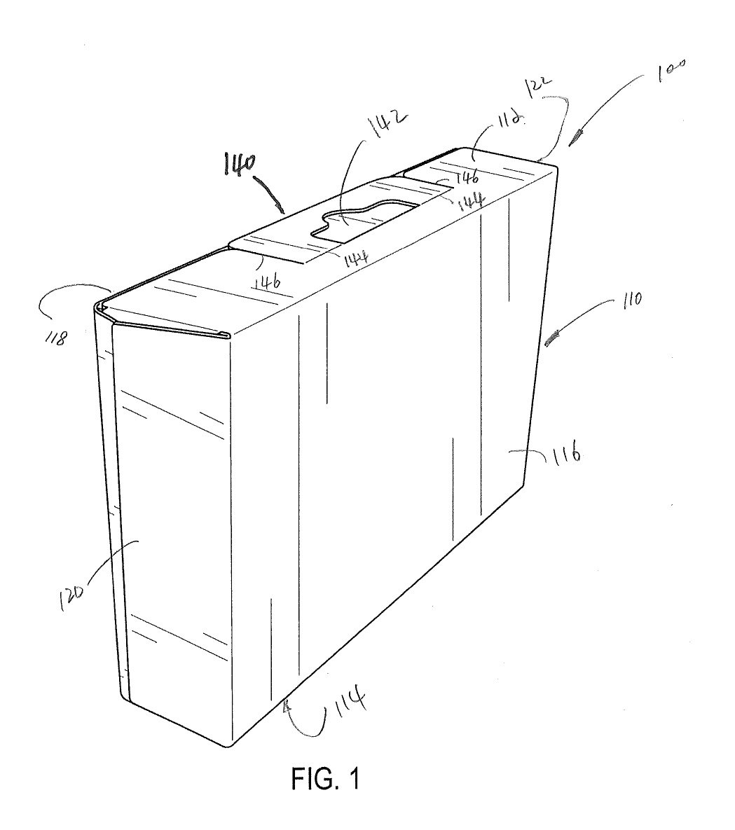

FIG. 1 is a perspective view of a package 100 according to an exemplary embodiment of the present invention. The package 100 is suitable for transporting, storing, and displaying various products, such as, for example, medical products, and displaying information related to the products. While the exemplary embodiments of the present invention are described with reference to the package 100 containing a medical product, those of skill in the art will understand that the package 100 may contain any number of products or types of products.

As shown, in the exemplary embodiment, the package 100 includes a body 110 for containing a medical product, and a tab 140 attached to the body 110 that is pivotable or foldable with respect to the body 110 between first and second positions.

In the exemplary embodiment shown in FIG. 1, the body 110 is substantially in the shape of a box, having a top panel 112 and an opposite bottom panel 114, a front panel 116 and an opposite rear panel 118, and a left panel 120 and an opposite right panel 122. The panels 112-122 are connected (e.g., in a single blank) to form, when folded together, an enclosed structure defining a space therein for containing the medical product. When folded into a box, the panels 112-122 can be sealed (e.g., with a suitable adhesive or via tabs and slots) to prevent ingress of dirt, dust, debris, and/or light into the body 110 for a satisfactory storage of the medical product in the package 100. Information about the medical product and/or artwork (e.g., trade names, logos, etc.) can be printed or otherwise presented on one or more of the panels 112-122.

In the shown embodiment, the body 110 is substantially in a shape of a box, with the opposite panels being similarly dimensioned and substantially planar and parallel to each other. However, certain variations and permutations of the shape and profile of the body as well as the panels are within the scope of the present invention. For example, the front panel may be concave and the rear panel may be convex to provide a nestable feature for the package, such that, when a plurality of packages is stacked, they can be nested together. Additionally, the front panel may include a sealed window, which allows the medical product to be seen by a consumer.

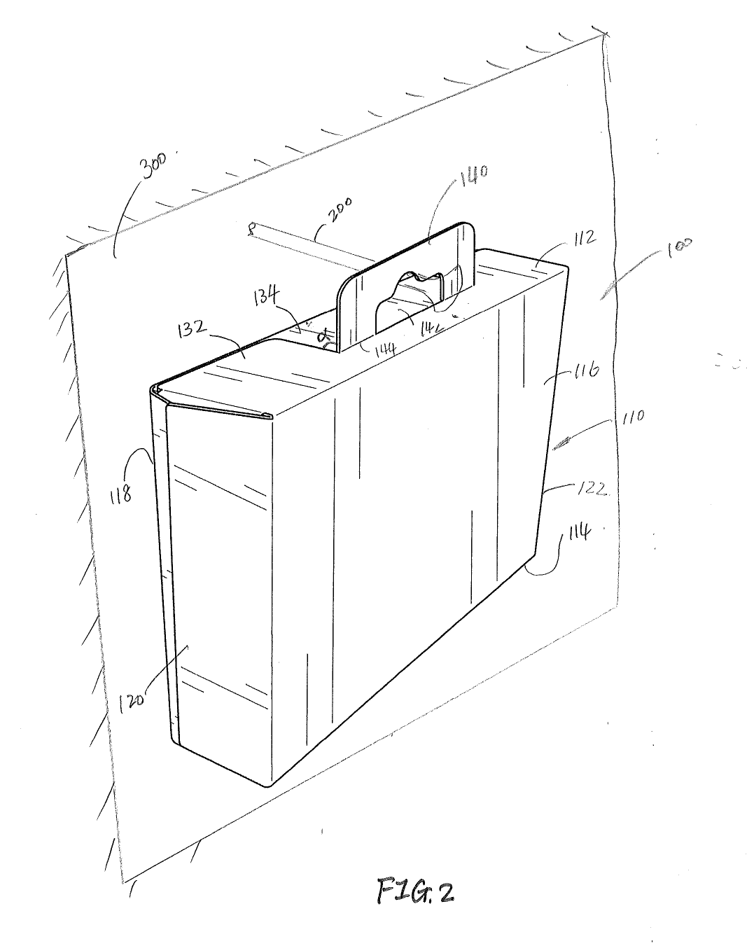

As shown in both FIG. 1 and FIG. 2, the tab 140 may be attached to the top panel 112 and pivotable between first and second positions with respect to the top panel 112. As understood by those of skill in the art, the tab 140 may be cut from the top panel 112. The tab 140 has an opening 142 formed therein, which extends through the tab 140. In the exemplary embodiment, the tab 140 is attached to the top panel 112 through a pair of hinges 144, disposed at either side of the opening 142, and formed integrally with the top panel 112. In the shown embodiment, the hinges are in the form of a preformed crease or score, which allows the tab 140 to pivot/fold with respect to the top panel 112. The location of the hinge 144 on the top panel 112 can be adjusted to, for example, adjust the dimensions of the tab 140. The opening 142 may include a filler element (not shown) formed therein, and a periphery of the opening 142 may include one or more perforations for removing the filler element when using the tab 140. Those of skill in the art will understand that the tab 140 may be formed from any of the panels and, along with the opening 142, may be any size and/or shape.

FIG. 1 illustrates a first position of the tab 140, where the tab 140 is substantially coplanar with the top panel 112, such that the tab 140 does not protrude from the top panel 112. Thus, when the tab 140 is in the first position, the package 100 is suitable for transportation, storage and display so that in particular the waste of space in a transportation vehicle, warehouse or on a shelf in a store caused by the protruding tabs and the interference between the tabs of adjacent packages can be avoided.

A confining mechanism 146 can be provided to maintain the tab 140 in the first position. In the shown embodiment, the confining mechanism 146 may include a pair of pre-perforated seams 146 bordering lateral sides of the tab 140. The pre-perforated seams 146 provide connection between the tab 140 and the top panel 112, while still allowing a user handling the package 100 to break the connection between the tab 140 and the top panel 112 and simultaneously pivot the tab 140 from the first position to a second position shown in FIG. 2. However, a person or ordinary skill in the art understands that other suitable means, such as a tape or adhesive, can be used in place of and/or in addition to the pre-perforated seams. For example, a thin layer of adhesive may be applied to an underside of the tab 140 to prevent it from becoming dislodged or folding inadvertently during transportation, packaging, etc. The tab 140 may further include, for example, one or more rounded or foldable edges which allow the user to detach the tab 140 from the seams 146 and pivot the tab 140 to the second position. In the shown embodiment, an edge is formed at a terminal end of each of the seams 146 opposite the hinge 144. The edge may provide a discontinuous border with a top edge of the top panel 112, allowing the user to easily grasp and separate the tab 140 from the seams 146 when pivoting it to the second position.

At the second position shown in FIG. 2, the tab 140 is deployed and forms an angle a with the top panel 112, such that the tab 140 can engage a supporting structure 200 through the opening 142. For example, the supporting structure 200 can be a peg for pegging the package 100 to a board or wall 300. Alternatively, the supporting structure 200 can be a rod to hang the package 100, such that the package 100 is slidable along the rod. For example, the tab 140 can be substantially perpendicular to the top panel 112, with the angle a being around 90 degrees.

As shown in FIG. 2, the top panel 112 may include an upper layer 132 and a lower layer 134. In the shown embodiment, the tab 140 may be formed in the upper layer 132. For example, in an exemplary embodiment, the opening 142 is formed in the upper layer 132 by cutting out the filler element corresponding to the dimensions of the opening 142; the hinges 144 are formed at either side of the opening 142 to enable pivoting of the tab 140; and the pre-perforated seams 146 are formed into the upper layer 132 depending on the dimensions of the tab 140. In this configuration, when the tab 140 is in the first position for packing, storage and/or transportation, a flat and continuous surface can be provided for the top panel 112 of the package 100.

When assembling the package 100, the top panel 112 may be formed by folding the upper layer 132 onto the lower layer 134. Further, adhesive may be applied to an underside of the upper layer 132 (except the tab 140) to secure it to the lower layer 134. For example, adhesive may be applied to the underside of sections of the upper layer 132 that are adjacent (e.g., lateral and/or in front of) the tab 140. The adhesive may be applied to all, substantially all or predetermined portions of the underside of the upper layer 132. Thus, when the tab 140 is in the second position, there is no hole, void or space left in the top panel 112 that would expose the contents of the package 100 to the external environment, and the upper layer 132 is secured to the lower layer 134. Any hole, void or space in the package 100, therefore, may be evidence of tampering. Similarly, since the tab 140 may be integrally formed with the upper layer 132 via the hinges 144, the tab 140 may rip or become detached, and the contents of the package 100 will not be exposed to the external environment.

The features of the present invention as applied to various specific embodiments thereof have been shown and described. It will also be understood that various omissions, substitutions and changes in the form and details of the devices illustrated and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A package for a medical product, comprising:
a body comprising a plurality of panels, wherein at least one panel comprises a first layer and a second layer;
a tab formed integrally with the first layer, wherein the tab is pivotable via at least one hinge formed in the first layer from a first position substantially coplanar with the first layer to a second position where the tab is disposed at an angle to the first layer; and
an adhesive securing the first layer to the second layer.

2. The package according to claim 1, wherein the tab is connected to the first layer via a pair of hinges.

3. The package according to claim 1, wherein the at least one hinge comprises a score.

4. The package according to claim 1, wherein when the tab is in the first position, the tab is connected to the first layer by a confining mechanism.

5. The package according to claim 4, wherein the confining mechanism comprises at least one pre-perforated seam.

6. The package according to claim 1, wherein the first layer is substantially planar and the tab is substantially planar.

7. The package according to claim 1, wherein the plurality of panels are connected to provide an enclosed structure defining a space therein.

8. The package according to claim 7, wherein when the tab is in the first and second positions, the space is not accessible from an environment external to the enclosed structure.

9. The package according to claim 1, wherein the tab comprises an opening formed therein.

10. The package according to claim 9, wherein the opening comprises a filler element.

11. The package according to claim 10, wherein a periphery of the opening is perforated to enable removal of the filler element.

12. (canceled)

13. The package according to claim 1, wherein the first layer comprises an adhesive applied to an underside thereof for adhering to an upper surface of the second layer.

14. The package according to claim 13, wherein the adhesive covers substantially all of the underside of the first layer.

15. The package according to claim 13, wherein the adhesive covers predetermined portions of the underside of the first layer.
